(19) United States
(12) Patent Application Publication
(10) Pub. No.: US 2024/0150693 A1
(43) Pub. Date: May 9, 2024

(54) STERILE CONTAINER FOR CELL CULTURE

(71) Applicant: SUMITOMO BAKELITE CO., LTD., Shinagawa-ku, Tokyo (JP)

(72) Inventor: Kohta Igarashi, Akita-shi (JP)

(21) Appl. No.: 18/277,591

(22) PCT Filed: Mar. 8, 2022
(86) PCT No.: PCT/JP2022/010012

§ 371 Date: Aug. 17, 2023

(30) Foreign Application Priority Data
Mar. 16, 2021 (JP) 2021-042041

(51) Int. Cl. C12M 1/32; C12M 1/00; C12M 1/12

(52) U.S. Cl. CPC C12M 23/12; C12M 23/20; C12M 37/00

(57) ABSTRACT

The invention of the present application provides a sterile container for cell culture that is sufficiently sterilized but sufficiently retains a low cell adsorption property and that easily forms cell aggregates by cell culture. The sterile container for cell culture having a low cell adsorption property of the invention of the present application includes a cell accommodation part which is made of a resin material and is capable of accommodating cells and a liquid culture medium to perform cell culture, in which a coating layer made of a water-soluble resin having a hydrophilic functional group at a side chain is formed on an inner surface of the cell accommodation part, cross-linking is formed in the water-soluble resin itself and between the water-soluble resin and the inner surface of the cell accommodation part, and the container is subjected to a radiation sterilization treatment with an absorbed radiation dose of 20 kGy or more and 45 kGy or less.

### TECHNICAL FIELD

The present invention relates to a sterile container for cell culture.

### BACKGROUND ART

As sterile containers for cell culture, which are containers used for cell culture, containers made of a resin material are often used. However, cells may be easily adsorbed to the inner surfaces of these containers made of a resin material. When cells are adsorbed to the inner surface of the container during cell culture, it is difficult to recover cells from inside the container and there is a possibility that the recovery rate thereof may decrease. In addition, the formation of cell aggregates (lumps formed by the three-dimensional aggregation of cells), which is necessary in fields such as regenerative medicine, tends to be difficult.

In order to solve these problems, methods are known for treating (with a low-adsorption treatment) the inner surfaces of biochemical containers such that cells and the like are not easily adsorbed thereto. For example, PTL 1 discloses a method for reducing the adsorption of proteins and the like on the surface of a member made of polystyrene, in which the member made of polystyrene is placed in a reaction container and ozone gas is distributed therein to introduce oxygen to the surface and thereby impart a hydrophilic property thereto. Other than the above, methods for coating the inner surface of a container with a hydrophilic polymer and the like are also known.

### CITATION LIST

### Patent Literature

[PTL 1] Japanese Unexamined Patent Application, First Publication No. H10-101820

### SUMMARY OF INVENTION

### Technical Problem

In recent years, in the field of regenerative medicine (for example, culturing ES cells, iPS cells, mesenchymal stem cells (MSC), and the like), there is an increasing demand for sterile containers for cell culture to have a high level of sterility. In such a case, there is a need for containers subjected to a stronger sterilization treatment; however, for containers for cell culture made of a resin material, the heat resistance of the resin material is a problem in dry heat sterilization or wet heat sterilization and the cytotoxicity of residues is a problem in sterilization using chemical substances such as ethylene oxide gas (EOG) and thus it was necessary to perform this strong sterilization treatment by radiation sterilization. However, as there was a concern that subjecting containers for cell culture to the low-adsorption treatment described above might destroy the hydrophilic groups and polymers and thereby decrease the low cell adsorption property or the like, there was a problem in that performing strong radiation sterilization (radiation sterilization at high doses) was difficult.

The present invention was made in consideration of the above-mentioned problems and has an object of providing a sterile container for cell culture that is sufficiently sterilized but retains a low cell adsorption property and that easily forms cell aggregates by cell culture.

### Solution to Problem

A sterile container for cell culture according to the present invention is a sterile container for cell culture having a low cell adsorption property, including a cell accommodation part which is made of a resin material and is capable of accommodating cells and a liquid culture medium to perform cell culture, in which a coating layer made of a water-soluble resin having a hydrophilic functional group at a side chain is formed on an inner surface of the cell accommodation part, cross-linking is formed in the water-soluble resin itself and between the water-soluble resin and the inner surface of the cell accommodation part, and the container is subjected to a radiation sterilization treatment with an absorbed radiation dose of 20 kGy or more and 45 kGy or less.

### Advantageous Effects of Invention

According to the present invention, it is possible to obtain a sterile container for cell culture that is sufficiently sterilized but sufficiently retains a low cell adsorption property and that easily forms cell aggregates by cell culture.

### BRIEF DESCRIPTION OF DRAWINGS

FIG. 1 is a schematic perspective view of a sterile container for cell culture (a multi-well plate) according to the present embodiment.

FIG. 2 is a schematic enlarged cross-sectional view of a cell accommodation part of the sterile container for cell culture (the multi-well plate) according to the present embodiment.

FIG. 3 is a schematic enlarged cross-sectional view showing the enlarged vicinity of an inner surface of the cell accommodation part of the sterile container for cell culture according to the present embodiment.

FIG. 4 is a schematic enlarged cross-sectional view of a state in which a cell aggregate is formed in the cell accommodation part of the sterile container for cell culture (the multi-well plate) according to the present embodiment.

### DESCRIPTION OF EMBODIMENTS

A description will be given below of an embodiment of the sterile container for cell culture according to the present invention based on the drawings.

The embodiment described below is only an example to facilitate understanding of the present invention and does not limit the present invention. That is, the shape, dimensions, arrangement, and the like of the members described below may be changed or improved without departing from the gist of the present invention and the present invention naturally includes equivalents thereof.

In addition, in all drawings, similar constituent components are indicated with similar reference numerals and duplicate explanations will not be repeated, as appropriate. For convenience, there are parts in some of the drawings not indicated by reference numerals (reference numerals are not used). Furthermore, the dimensional proportions of each member shown in the drawings may differ from the actual dimensional proportions in order to facilitate understanding of the invention.

### Overview

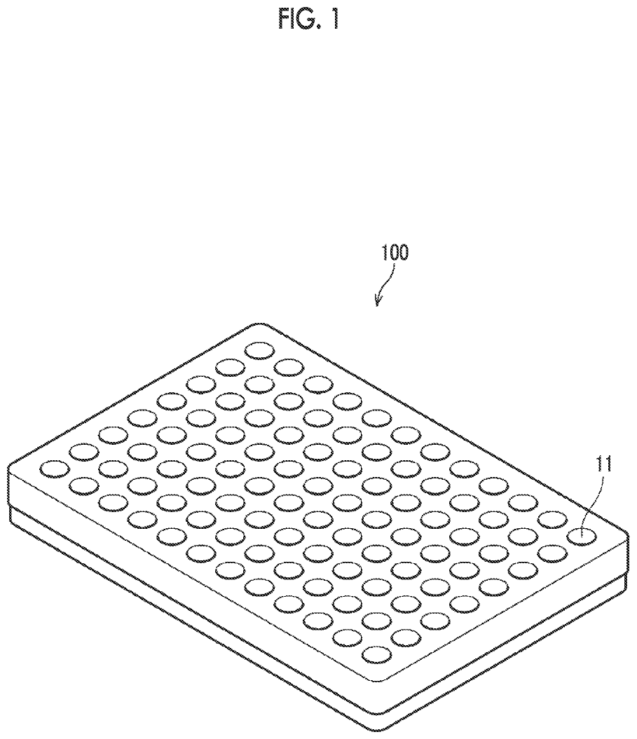

First, a detailed description will be given of an overview of the sterile container for cell culture according to the present embodiment with reference to FIG. 1. FIG. 1 is a perspective view schematically showing a sterile container for cell culture 100 according to the present embodiment.

The sterile container for cell culture 100 according to the present embodiment is the sterile container for cell culture 100 having a low cell adsorption property, including a cell accommodation part 11, which is made of a resin material and is capable of accommodating cells and a liquid culture medium to perform cell culture, in which a coating layer 21 made of a water-soluble resin having a hydrophilic functional group at a side chain is formed on an inner surface 11a of the cell accommodation part 11, cross-linking is formed in the water-soluble resin itself and between the water-soluble resin and the inner surface 11a of the cell accommodation part 11, and the container is subjected to a radiation sterilization treatment with an absorbed radiation dose of 20 kGy or more and 45 kGy or less. “Low cell adsorption property” means that, under the same culturing conditions, the ratio of cell adsorption is lower than that of a sterile container for cell culture made of a resin material and not subjected to a surface treatment (also including coating layer formation). In addition, the “inner surface 11a of the cell accommodation part 11” is the surface of the cell accommodation part 11 (for example, a well or the like) of the sterile container for cell culture 100 according to the present embodiment on the side of the internal space able to accommodate a liquid culture medium and the like.

First, in the sterile container for cell culture 100 according to the present embodiment, it is sufficient if at least the cell accommodation part 11 is made of a resin material. Here, “made of a resin material” means that the resin material is included as the main material (the resin material is included as 70% by mass or more of the total mass, more preferably 80% by mass or more, and even more preferably 90% by mass or more) and materials other than the resin material, such as additives, may only be included in a range not influencing the effect of the present invention. The sterile container for cell culture 100 according to the present embodiment may be entirely (all the provided members) made of the resin material, or at least a part of the members other than the cell accommodation part 11 may be made of a material (for example, glass or the like) other than the resin material; however, it is more preferable that the sterile container for cell culture 100 according to the present embodiment is entirely made of a resin material (in particular, the same resin material). This is because it is easier to manufacture (by injection molding or the like) the sterile container for cell culture 100 according to the present embodiment.

As these resin materials, it is possible to use, for example, polyolefin-based resins or cyclic polyolefin-based resins such as polypropylene resin, polyethylene resin, polymethylpentene resin, and ethylene-propylene copolymers, polystyrene-based resins such as polystyrene and acrylonitrile-butadiene-styrene resins, polyacrylic-based resins such as polyethylene terephthalate resin and polybutylene terephthalate resin, methacrylic-based resins such as polymethyl methacrylate resin, fluorine-based resins such as polytetrafluoroethylene, acrylic-based resins such as polyacrylonitrile, fiber-based resins such as propionate resin, polycarbonate resin, vinyl chloride resin, polyarylate resin, polysulfone resin, polyethersulfone resin, polyetheretherketone resin, polyetherimide resin, and the like. The above may be used alone as one type, or two or more types may be used in combination. Among the above, it is particularly preferable to use polystyrene-based resins from the point of view of moldability, transparency, radiation resistance (sterilizability), and the like, which are demanded for containers for cell culture.

In addition, the weight average molecular weight of the resin material is not particularly limited, but 10,000 or more and 500,000 or less is preferable and 20,000 or more and 100,000 or less is more preferable. This is because the moldability is superior when the weight average molecular weight of the resin material to be used is in this range.

The weight average molecular weight is a value measured by the size exclusion chromatography method (Gel Permeation Chromatography system, Shodex KF-800 column, both manufactured by Showa Denko K.K., elution solvent: tetrahydrofuran).

Using such a resin material as the main material, it is possible to form the cell accommodation part 11 of the sterile container for cell culture 100 according to the present embodiment by, for example, injection molding, blow molding, injection blow molding, vacuum molding, or the like and it is also possible to form the entire sterile container for cell culture 100 according to the present embodiment.

The form of the sterile container for cell culture 100 according to the present embodiment is not particularly limited as long as it is provided with the cell accommodation part 11 having an internal space which is capable of accommodating cells and a liquid culture medium to perform cell culture and examples thereof include a multi-well plate as shown in FIG. 1, a petri dish (dish), a flask made of resin, and the like. However, other forms which are provided with the cell accommodation part 11 and are able to be installed and used in an environment where cells are able to be cultured may be used. Among the above, from the viewpoint of being able to improve the accuracy of evaluation and research using cell aggregates, multi-well plates and petri dishes used for growth carried out in bioreactors, evaluation of medicinal effects and toxic substances, artificial organ development research, and the like are preferable. A multi-well plate is the sterile container for cell culture 100 provided with a plurality of wells, which are the cell accommodation parts 11 and suitable examples may be provided with 6, 12, 24, 48, 96, or 384 wells. This is because it is possible to perform the cell culture simultaneously under the same conditions in a plurality of wells.

In addition, the volume of the cell accommodation parts 11 (the volume of the internal space able to accommodate a liquid culture medium and the like) is also not limited, but, for example, the volume of one of the cell accommodation parts 11 may be 0.05 mL or more or may be 0.1 mL or more. The upper limit is also not limited, but may be 20 mL or less or may be 16 mL or less.

The sterile container for cell culture 100 according to the present embodiment may further include other members (for example, a lid material or the like) in addition to the member including the cell accommodation parts 11. In addition, a gripping portion able to be gripped by a hand may be provided in the member or the like including the cell accommodation parts 11.

The sterile container for cell culture 100 according to the present embodiment is suitable for use as a sealed and packaged sterile container for cell culture that is sealed and packaged using two or more layers, more preferably three or more layers, using a gas permeable film material (all film materials in the multi-layer packaging are gas permeable). This is because it is possible to obtain a container in which the configuration is suitable for delivery and use in cell processing facilities (CPF) and cell processing centers (CPC), or the like and the residual radiation odor, which may be generated by the radiation sterilization treatment described below, in the sealed packaging is low. In particular, gas permeable and heat sealable film materials are suitable due to being easier to seal and package. Such film materials are not limited, but examples thereof include film materials or the like made of polyethylene (for example, HDPE, LDPE, LLDPE, and the like) or polypropylene. For the film material, the gas permeability measured under conditions of a temperature of 25° C. and a relative humidity (RH) of 0% according to JIS K7126-1 is more preferably 0.1 L/m² day atm or higher, even more preferably 0.5 L/m² day atm or higher, and yet more preferably 1.0 L/m² day atm or higher.

### Configuration of Cell Accommodation Part

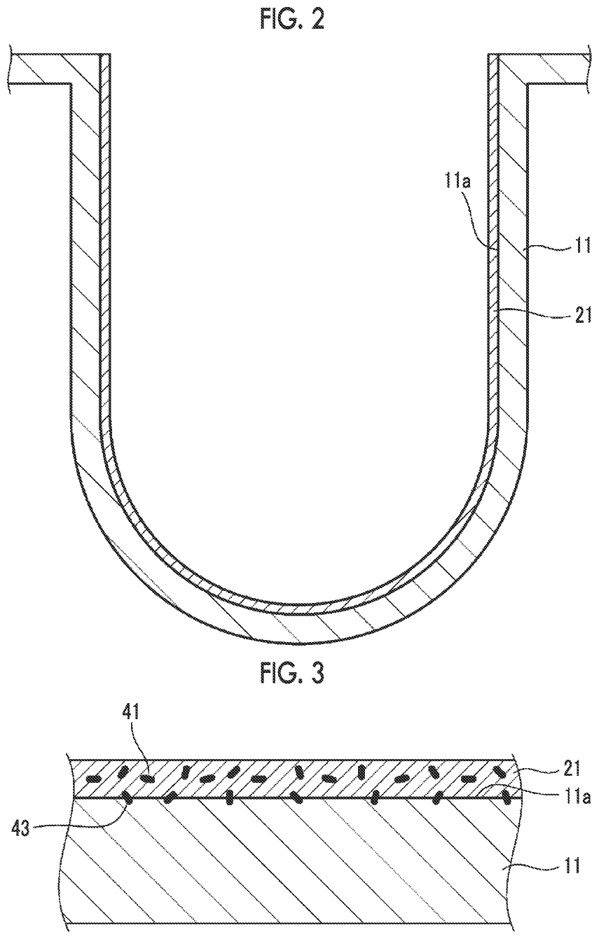

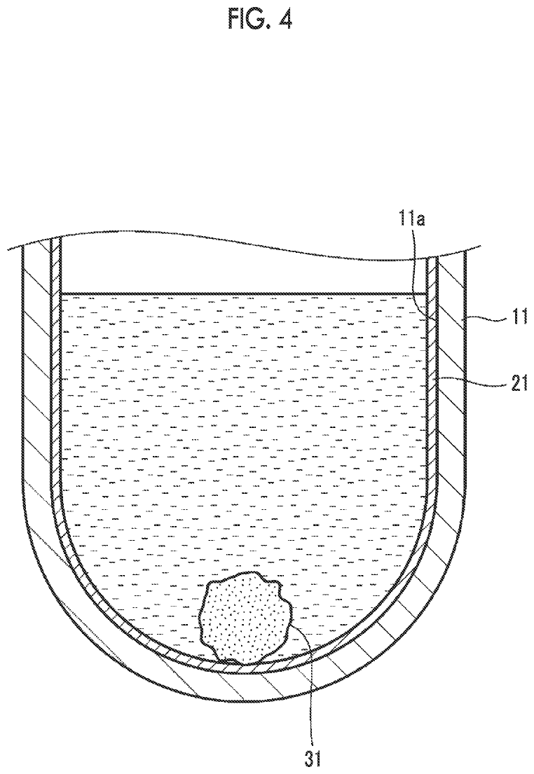

Next, a detailed description will be given of the configuration of the cell accommodation part 11 of the sterile container for cell culture 100 according to the present embodiment with reference to FIG. 2 to FIG. 4. FIG. 2 is an enlarged cross-sectional view schematically showing the cell accommodation part 11 of the sterile container for cell culture 100 according to the present embodiment. In addition, FIG. 3 is a schematic enlarged cross-sectional view showing the further enlarged vicinity of the inner surface 11a of the cell accommodation part 11 of the sterile container for cell culture 100 according to the present embodiment. Furthermore, FIG. 4 is a schematic enlarged cross-sectional view showing a state in which a cell aggregate 31 is formed in the cell accommodation part 11 of the sterile container for cell culture 100 according to the present embodiment.

In the sterile container for cell culture 100 according to the present embodiment, the coating layer 21 made of a water-soluble resin having a hydrophilic functional group at a side chain is formed on the inner surface 11a of the cell accommodation part 11, as in the embodiment shown in FIG. 2, for example. As shown in FIG. 3, cross-linking is formed both in the water-soluble resin itself, that is, between the water-soluble resin molecules in the coating layer 21, and between the water-soluble resin (the water-soluble resin molecules in the coating layer 21) and the inner surface 11a of the cell accommodation part 11. By forming the coating layer 21, a hydrophilic functional group is included in the surface layer of the inner surface 11a of the cell accommodation part 11 and cells accommodated in the cell accommodation part 11 are not easily adsorbed to the container. Due to this, cells easily gather in the vicinity of the bottom of the cell accommodation part 11 during culturing, making a container in which the cell aggregate 31 is easily formed, as shown in FIG. 4.

The “water-soluble resin” in the present embodiment is a resin dissolvable in water by hydration through ionic bonding or hydrogen bonding with water molecules and of which it is possible to dissolve 1.0 g or more per 100 g of water at 25° C. In addition, the term “coating layer 21 made of a water-soluble resin” means the coating layer 21 including the water-soluble resin described above as a main component (for example, including 80% by mass or more or even 90% by mass or more) and encompasses a layer in which the water-soluble resin included in the coating layer 21 is cross-linked and cured such that the coating layer 21 is modified to be non-water-soluble. In other words, when the coating layer 21 is formed using a material in which a water-soluble resin is the main component, cross-linking and curing may be carried out to make the coating layer 21 non-water-soluble.

Examples of water-soluble resins having hydrophilic functional groups at a side chain include a saponide of polyvinyl acetate, polyvinyl pyrrolidone, polyethylene glycol, polyacrylamide, polymethacrylamide, polyhydroxyethyl methacrylate, polypentaerythritol triacrylate, polypentaerythritol tetraacrylate, poly(diethylene glycol) diacrylate, and copolymers of the monomers forming the above, copolymers of 2-methacryloyloxyethyl phosphorylcholine and other monomers (for example, butyl methacrylate or the like), and the like. These water-soluble resins also encompass resins in which another hydrophilic functional group or the like is added to a part thereof as a side chain. Among the above, one or more selected from a saponide of polyvinyl acetate, polyvinyl pyrrolidone, and polyethylene glycol is more preferable. This is because it is possible to suppress stimuli with respect to the cells as a result. The average degree of polymerization of the water-soluble resin is not particularly limited; however, 100 to 10,000 is preferable and 200 to 5,000 is more preferable since it is easy to form a uniform film on the inner surface 11a of the cell accommodation part 11 and the workability is favorable.

Examples of saponides of polyvinyl acetate include a copolymer of polyvinyl alcohol or vinyl alcohol and other compounds, or a saponide of a modified vinyl acetate, which is modified with hydrophilic group modification, hydrophobic group modification, anionic modification, cationic modification, amide group modification, or modification with a reaction group such as an acetoacetyl group, and vinyl alcohol, or the like. The degree of saponification of the saponide of polyvinyl acetate is not particularly limited; however, 20 mol % to 100 mol % of the whole polyvinyl acetate is preferable and 50 mol % to 95 mol % is more preferable.

The water-soluble resin forming the coating layer 21 has a hydrophilic functional group at a side chain, but may have a functional group for further cross-linking and curing, or the functional group for cross-linking and curing may be a hydrophilic functional group or a part thereof. Examples of these functional groups include functional groups including hydroxyl groups and carbonyl groups (carboxy groups, aldehyde groups, ketone groups, ester groups, amide groups, and the like), radiation-reactive, photosensitive, and thermally reactive functional groups, or the like. Furthermore, more preferable examples thereof include functional groups including diazo groups, azide groups, and diazide groups, which are photosensitive hydrophilic functional groups.

In particular, due to being easily cross-linked and curable by light irradiation at a wavelength of 300 to 500 nm, having high stability against a radiation sterilization treatment as described below, and further making it possible to reduce the amount of cells adsorbed, water-soluble resins having functional groups including azide groups and/or carbonyl groups at the side chain are more preferable, water-soluble resins having functional groups including aromatic azide groups and/or carbonyl groups at the side chain are even more preferable, and water-soluble resins represented by Formula (Ia) or Formula (Ib) are yet more preferable.

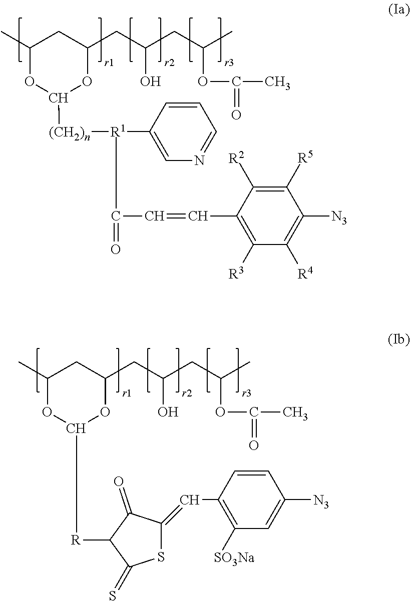

In Formula (Ia), R1 is a hydrocarbon group having a carbonyl and an amine and may include an amide bond, R2, R3, R4, and R5 are all hydrogen or alkyl groups, r1 is 1 to 1000, r2 is 40 to 4995, r3 is 0 to 4000, and n is 1, 2, or 3. In particular, R2, R3, R4, and R5 are more preferably all hydrogen; however, by making at least one of R2, R3, R4, and R5 an alkyl group, it is possible to impart hydrophobicity to a part of the water-soluble resin, such that it is possible to expect an effect of further improving the adhesiveness of the water-soluble resin to the container.

In addition, in Formula (Ib), R indicates a hydrocarbon group having a carbonyl and an amine, r1 indicates 1 to 1000, r2 indicates 40 to 4995, and r3 indicates 0 to 4000.

Furthermore, the water-soluble resin represented by Formula (Ia) is even more preferably a water-soluble resin represented by Formula (IIa).

In Formula (IIa), R indicates an alkyl group having a carbonyl and an amine and may include an amide bond, r1 indicates 1 to 1000, r2 indicates 40 to 4995, r3 indicates 0 to 4000, and n indicates 1, 2, or 3.

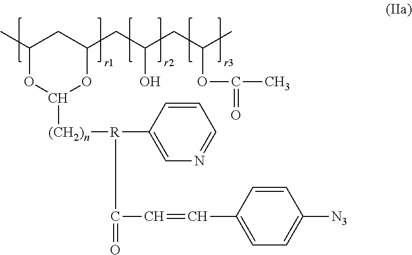

The coating layer 21 is formed by coating the water-soluble resin described above on the inner surface 11a of the cell accommodation part 11 and cross-linking and curing the molecules of the water-soluble resin included in the coating layer 21 by light irradiation or the like. In other words, in this configuration, the water-soluble resin in the coating layer 21 is cross-linked to itself (for example, a cross-linked structure 41 in FIG. 3). This cross-linking is preferably formed by the functional groups of the side chains of the water-soluble resin. A part of the coating layer 21 may include molecules of the water-soluble resin that are not cross-linked as long as the cross-linking and curing is carried out to the extent that it is possible to maintain the structure as the coating layer 21. Due to this, even after strong radiation sterilization, it is possible to obtain a container with a low amount of eluted material from the coating layer 21 when cells, a liquid culture medium, and the like are accommodated in the cell accommodation part 11 and the low cell adsorption property of the coating layer 21 is maintained for a long period of time. Here, the cross-linked structure 41 in FIG. 3 is an easy-to-understand representation of the cross-linked structure between molecules of the water-soluble resin and does not represent the actual size or number of cross-linked structures.

Furthermore, in the sterile container for cell culture 100 according to the present embodiment, the cross-linking is formed not only in the water-soluble resin forming the coating layer 21, but also between the water-soluble resin forming the coating layer 21 and the inner surface 11a of the cell accommodation part 11 (for example, a cross-linked structure 43 in FIG. 3). This cross-linking is preferably formed by the functional groups of the side chains of the water-soluble resin and the functional groups of the inner surface 11a of the cell accommodation part 11 (functional groups formed by the surface treatment described below or the like). Due to this, the coating layer 21 is highly adhesive to the inner surface 11a of the cell accommodation part 11. In other words, the stability of the coating layer 21 is further increased. Here, the cross-linked structure 43 in FIG. 3 is also an easy-to-understand representation of the cross-linked structure between the molecules of the water-soluble resin and the inner surface 11a of the cell accommodation part 11 and also does not represent the actual size or number of cross-linked structures.

The inner surface 11a of the cell accommodation part 11, where the coating layer 21 is formed, may be subjected to a surface treatment such as a hydrophilic treatment or a treatment to form surface treatment functional groups on the surface before the formation of the coating layer 21. Examples of such a surface treatment include a treatment for forming an oxygen-containing functional group. By forming the oxygen-containing functional group on the surface, the above-described cross-linking (cross-linking with the water-soluble resin of the coating layer 21, the cross-linked structure 43 in FIG. 3) is more easily formed. Examples of oxygen-containing functional groups include polar functional groups such as carbonyl groups (carboxy groups, aldehyde groups, ketone groups, ester groups, and the like), hydroxyl groups, ether groups, peroxide groups, and epoxy groups. In particular, it is more preferable to carry out a treatment for forming a carbonyl group as an oxygen-containing functional group. In addition, as this surface treatment, for example, it is possible to adopt a plasma treatment, a corona discharge treatment, an excimer laser treatment, a flame treatment, or the like and it is particularly suitable to perform a plasma treatment.

The amount of the oxygen-containing functional groups formed by the above-described treatment is not particularly limited, but 0.01 to 100 nmol/cm² is preferable and 0.05 to 10 nmol/cm² is particularly preferable. When the amount of oxygen-containing functional groups is less than this lower limit value, there is a possibility that the desired effect may not be sufficiently exhibited and, when the amount exceeds the upper limit value, there is a possibility that the characteristics of the inner surface 11a itself of the cell accommodation part 11 may change and the performance demanded as a container for cell culture may not be achieved.

Here, in a suitable configuration, the water-soluble resin forming the coating layer 21 has a side chain including an aromatic azide group and a side chain including a carbonyl group, the carbonyl group is formed in the inner surface 11a of the cell accommodation part 11, and cross-linking is formed between the aromatic azide group of the water-soluble resin and the carbonyl group of the water-soluble resin, as well as between the aromatic azide group of the water-soluble resin and the carbonyl group of the inner surface 11a of the cell accommodation part 11. This is because these cross-links are extremely easy to form and it is easy to form the coating layer 21 which is more stable with respect to the radiation sterilization treatment described below.

In addition, the thickness of this coating layer 21 is not particularly limited, but, for example, 0.1 μm to 50 μm is preferable and 0.5 μm to 20 μm is more preferable. The thickness of the coating layer 21 is preferably uniform on the inner surface 11a of the cell accommodation part 11. Here, the “thickness of the coating layer 21” is the length of the shortest distance from the surface of the coating layer 21 (the surface on the internal space side of the cell accommodation part 11) to the surface in contact with the inner surface 11a of the cell accommodation part 11. In addition, the thickness being “uniform” means that, when the thickness of the coating layer 21 formed on the inner surface 11a of the cell accommodation part 11 is measured at 10 arbitrary locations, the variation (difference between the maximum and minimum) of the measured values is within 0.1 μm and more preferably within 0.05 μm.

The thickness of the coating layer 21 is measured by an ellipsometer.

Since the sterile container for cell culture 100 according to the present embodiment, in which the coating layer 21 as described above is formed on the inner surface 11a of the cell accommodation part 11, is sufficiently sterilized but cells are not easily adsorbed to the inner surface 11a of the cell accommodation part 11 due to the coating layer 21 and the cell aggregate 31 as shown in FIG. 4 is easily formed by cell culture, the sterile container for cell culture 100 is extremely suitable for use in the field of regenerative medicine and the like. A detailed description will be given below of the sterilization treatment (radiation sterilization treatment) of the sterile container for cell culture 100 according to the present embodiment.

### Radiation Sterilization Treatment

The sterile container for cell culture 100 according to the present embodiment is a container provided with the cell accommodation part 11 on which the coating layer 21 as described above is formed, which is subjected to a radiation sterilization treatment with an absorbed radiation dose of 20 kGy or more and 45 kGy or less. This radiation is preferably γ-rays or electron beams and, in the case of mass production or the case of the above-described multi-layer packaging or the like, γ-ray sterilization is particularly preferable in terms of radiation permeability. In addition, the absorbed radiation dose is more preferably 22.5 kGy or more and 40 kGy or less. This configuration provides a high level of sterility and the predetermined coating layer 21 is maintained in a stable state, thus, the low cell adsorption property is retained and cell aggregates are easily formed by cell culture.

In other words, this coating layer 21 has a feature of not easily decomposing even when subjected to the radiation sterilization treatment as described above and having a small amount of eluted material from the coating layer 21 in the liquid culture medium and the like. For example, it is possible to set the sterile container for cell culture 100 according to the present embodiment such that, while sterilization is carried out sufficiently, when pure water is accommodated in the cell accommodation part 11 to be 0.12 mL/cm² and heating is carried out at 37° C. for 72 hours, the obtained test solution is placed in a cell with a layer length of 1 cm, the absorbance measured by irradiation with UV light having a wavelength of 220 nm and the absorbance measured by irradiation with UV light having a wavelength of 241 nm are both 0.10 or less. In other words, it is possible to reduce decomposition of the coating layer 21 even when the strong radiation sterilization treatment as described above is carried out. In the sterile container for cell culture 100 according to the present embodiment, the absorbance measured by irradiation with UV light having a wavelength of 220 nm as described above may be greater than 0.06 and may be 0.07 or more. In addition, the absorbance measured by irradiation with UV light having a wavelength of 241 nm as described above may be greater than 0.03 and may be 0.04 or more.

The amount of pure water described above is the amount with respect to the surface area of the region where the coating layer 21 is formed on the inner surface 11a of the cell accommodation part 11, which accommodates pure water. In addition, the absorbance described above is measured by a UV spectrophotometer.

The sterile container for cell culture 100 according to the present embodiment has a high level of sterility and, furthermore, forming the cell aggregate 31 as shown in FIG. 4 by cell culture is extremely easy. For example, it is possible to obtain a container in which, in a case where human hepatocellular carcinoma-derived cells (HepG2) are accommodated in each of 96 of the cell accommodation parts 11 (a multi-well plate or the like) along with Dulbecco's modified Eagle's medium (DMEM) containing fetal bovine serum (FBS), the number of cells of the human hepatocellular carcinoma-derived cells (HepG2) in each one of the cell accommodation parts 11 is 1×10³ cells, and cell culture is performed at 37° C. for 3 days, the number of the cell accommodation parts 11 in which one or more of the cell aggregates 31 having a diameter of 200 μm or more of human hepatocellular carcinoma-derived cells (HepG2) are formed is 88 or more, 90 or more, 92 or more, or 95 or more.

As long as the sterile container for cell culture 100 according to the present embodiment has a high level of sterility and a low cell adsorption property, the radiation sterilization treatment as described above may be carried out, or a sterilization treatment other than the radiation sterilization treatment may be carried out. For example, the cell accommodation parts 11 and the like may be made of a highly heat-resistant resin material and subjected to dry heat sterilization or steam sterilization. In order to maintain the stability and the low cell adsorption property of the coating layer 21 at a high level and to easily form cell aggregates by cell culture, it is preferable to have a configuration that meets the conditions described above. That is, preferably, the sterile container for cell culture 100 is provided with the cell accommodation part 11, which is made of a resin material and is capable of accommodating cells and a liquid culture medium to perform cell culture, in which the coating layer 21 made of a water-soluble resin having a hydrophilic functional group at a side chain is formed on the inner surface 11a of the cell accommodation part 11, cross-linking is formed in the water-soluble resin itself and between the water-soluble resin and the inner surface 11a of the cell accommodation part 11, when pure water is accommodated in the cell accommodation part 11 to be 0.12 mL/cm² and heating is carried out at 37° C. for 72 hours, the obtained test solution is placed in a cell with a layer length of 1 cm, the absorbance measured by irradiation with UV light with a wavelength of 220 nm and the absorbance measured by irradiation with UV light at a wavelength of 241 nm are both 0.10 or less, and, in a case where human hepatocellular carcinoma-derived cells (HepG2) are accommodated in each of 96 of the cell accommodation parts 11 along with Dulbecco's modified Eagle's medium (DMEM) containing fetal bovine serum (FBS), the number of cells of the human hepatocellular carcinoma-derived cells (HepG2) in each of the cell accommodation parts 11 is 1×10³ cells, and cell culture is performed at 37° C. for 3 days, the number of the cell accommodation parts 11 in which one or more of the cell aggregates 31 having a diameter of 200 μm or more of human hepatocellular carcinoma-derived cells (HepG2) are formed is 88 or more, 92 or more, or 95 or more. The lower limit of the absorbance may be the same as described above.

### Method for Manufacturing Sterile Container for Cell Culture

Next, a description will be given of the method for manufacturing the sterile container for cell culture 100 according to the present embodiment.

In the method for manufacturing the sterile container for cell culture 100 according to the present embodiment, first, a container of a desired shape provided with the cell accommodation part 11 is molded by injection molding or the like, using a material made of the resin material described above. Then, the coating layer 21 is formed on the inner surface 11a of the cell accommodation part 11 of the obtained container using a material for which the water-soluble resin as described above is the main component. It is suitable to form the coating layer 21 after carrying out a surface treatment such as a plasma treatment in an oxygen atmosphere on the inner surface 11a of the cell accommodation part 11.

As a method for forming the coating layer 21 made of the water-soluble resin on the inner surface 11a of the cell accommodation part 11, for example, it is possible to use dipping or a method for dispensing a water-soluble resin solution into the cell accommodation part 11 and then tilting the container to discharge the solution, or the like. Using such a method, it is possible to form the coating layer 21 by bringing the water-soluble resin solution into contact with the inner surface 11a of the cell accommodation part 11 and then drying the residual solvent or the like.

The water-soluble resin in this case is suitably used as a solution prepared using a solvent such that the viscosity at 20° C. is preferably 1 mPa-s or more and 10 mPa-s or less and more preferably 2 mPa-s or more and 7 mPa-s or less. As the solvent at that time, it is possible to use water or a mixture of water and an organic solvent to increase solubility. When the viscosity of the water-soluble resin solution used is in the range described above, it is possible to easily obtain the coating layer 21 having an excellent low adsorption capacity for cells.

A step is performed in which cross-linking and curing are carried out in the water-soluble resin itself in the formed coating layer 21 and between the water-soluble resin and the inner surface 11a of the cell accommodation part 11 so as modify the coating layer 21 into a non-water-soluble cured film. It is possible to perform this step using a light irradiation treatment, a heat treatment, a radiation irradiation treatment, or the like as described above, which may be appropriately selected according to workability, the characteristics of the water-soluble resin forming the coating layer 21, the type of functional group formed on the inner surface 11a of the cell accommodation part 11, and the like. Among these, a light irradiation treatment (UV irradiation treatment or the like) is a method that makes it possible to perform the cross-linking and curing treatment quickly and with simple equipment.

In the case of cross-linking and curing the coating layer 21 by light irradiation, the light source is not particularly limited; however, it is possible to use a super high-pressure mercury lamp having an illuminance of approximately 5.0 mW/cm² or a UV lamp of approximately 0.1 mW/cm². Since it is possible to control the cross-linking and curing by light irradiation through the illuminance and irradiation time, the irradiation time may be lengthened in a case where a light source having a low illuminance is used and it is also possible to perform the cross-linking and curing under a fluorescent lamp in a case where a photosensitive group having high reactivity is selected. For example, irradiation for 1 to 10 seconds is sufficient to carry out the cross-linking and curing in a case of using a 5.0 mW/cm² super high-pressure mercury lamp and irradiation for 3 to 10 minutes is sufficient in a case of using a 0.1 mW/cm² UV lamp.

In this manner, by modifying the coating layer 21 into a non-water-soluble cured film, it is possible to construct the stable coating layer 21 having a hydrophilic functional group on the surface layer. After curing by cross-linking, the surface of the coating layer 21 may be washed with water or an aqueous solvent to remove at least a part of the unreacted water-soluble resin. By including such a washing step for the coating layer 21 after curing, it is possible to further reduce the amount of eluted material.

The sterile container for cell culture 100 according to the present embodiment, in which the coating layer 21 is formed as described above, is sealed and packaged using two or more layers using a film material having a gas permeability as necessary, further packed in a box or the like as necessary, and then the radiation sterilization treatment as described above is performed. In addition, a sterilization treatment other than the radiation sterilization treatment may also be performed as long as the configuration is as described above. Through the manufacturing method described above, it is possible to manufacture the sterile container for cell culture 100 according to the present embodiment, in which the coating layer 21 retains stability and has high sterility.

The embodiment described above encompasses the following technical ideas.
(1) A sterile container for cell culture having a low cell adsorption property, including a cell accommodation part which is made of a resin material and is capable of accommodating cells and a liquid culture medium to perform cell culture, in which a coating layer made of a water-soluble resin having a hydrophilic functional group at a side chain is formed on an inner surface of the cell accommodation part, cross-linking is formed in the water-soluble resin itself and between the water-soluble resin and the inner surface of the cell accommodation part, and the container is subjected to a radiation sterilization treatment with an absorbed radiation dose of 20 kGy or more and 45 kGy or less.
(2) The sterile container for cell culture according to (1), in which, when pure water is accommodated in the cell accommodation part to be 0.12 mL/cm², heating is carried out at 37° C. for 72 hours, and an obtained test solution is placed in a cell with a layer length of 1 cm, an absorbance measured by irradiation with UV light at a wavelength of 220 nm and an absorbance measured by irradiation with UV light at a wavelength of 241 nm are both 0.10 or less.
(3) The sterile container for cell culture according to (1) or (2), in which, when human hepatocellular carcinoma-derived cells (HepG2) are accommodated in each of 96 cell accommodation parts along with Dulbecco's modified Eagle's medium (DMEM) containing fetal bovine serum (FBS), the number of cells of the human hepatocellular carcinoma-derived cells (HepG2) in each of the cell accommodation parts is 1×10³ cells, and cell culture is performed at 37° C. for 3 days, the number of cell accommodation parts in which one or more cell aggregates having a diameter of 200 μm or more of the human hepatocellular carcinoma-derived cells (HepG2) are formed is 88 or more.
(4) The sterile container for cell culture according to any one of (1) to (3), in which the water-soluble resin forming the coating layer has a side chain including an aromatic azide group and a side chain including a carbonyl group, a carbonyl group is formed in the inner surface of the cell accommodation part, and cross-linking is formed between the aromatic azide group of the water-soluble resin and the carbonyl group of the water-soluble resin and between the aromatic azide group of the water-soluble resin and the carbonyl group in the inner surface of the cell accommodation part.
(5) The sterile container for cell culture according to any one of (1) to (4), in which the water-soluble resin forming the coating layer is a water-soluble resin represented by Formula (Ia) or Formula (Ib).
(6) The sterile container for cell culture according to any one of (1) to (5), in which the cell accommodation part is a well and the sterile container for cell culture is a multi-well plate provided with a plurality of the wells.
(7) A sealed and packaged sterile container for cell culture in which the sterile container for cell culture according to any one of (1) to (6) is sealed and packaged using two or more layers using a film material having a gas permeability.
(8) A sterile container for cell culture having a low cell adsorption property, including a cell accommodation part which is made of a resin material and is capable of accommodating cells and a liquid culture medium to perform cell culture, in which a coating layer made of a water-soluble resin having a hydrophilic functional group at a side chain is formed on an inner surface of the cell accommodation part, cross-linking is formed in the water-soluble resin itself and between the water-soluble resin and the inner surface of the cell accommodation part, when pure water is accommodated in the cell accommodation part to be 0.12 mL/cm², heating is carried out at 37° C. for 72 hours, and an obtained test solution is placed in a cell with a layer length of 1 cm, an absorbance measured by irradiation with UV light at a wavelength of 220 nm and an absorbance measured by irradiation with UV light at a wavelength of 241 nm are both 0.10 or less, and when human hepatocellular carcinoma-derived cells (HepG2) are further accommodated in each of 96 cell accommodation parts along with Dulbecco's modified Eagle's medium (DMEM) containing fetal bovine serum (FBS), the number of human hepatocellular carcinoma-derived cells (HepG2) is 1×10³ cells in each of the cell accommodation parts, and cell culture is performed at 37° C. for 3 days, the number of cell accommodation parts in which one or more cell aggregates having a diameter of 200 μm or more of the human hepatocellular carcinoma-derived cells (HepG2) are formed is 88 or more.

### EXAMPLES

A description will be given below of Examples of the present invention, but the present invention is not limited to the following Examples and various modifications are possible within the technical concept of the present invention.

### Example 1

A 96-well multi-well plate (width: 127.6 mm, length: 85.8 mm, height: 14.0 mm) as shown in FIG. 1 was molded by injection molding using polystyrene resin (manufactured by PS Japan Corporation, trade name HF77). The shape of the wells, which are the cell accommodation parts, was as shown in FIG. 2, the opening diameter of each well was 6.2 mm, the depth was 5.0 mm, the side wall thickness was 0.8 mm, and the volume per well was approximately 0.25 mL.

The obtained multi-well plates were subjected to a plasma treatment (10 minutes of oxygen plasma) in an oxygen atmosphere using a plasma treatment system (SERIES 7000 manufactured by Branson/IPC). Due to this, wettability was imparted to the plate surface.

Next, to perform a surface treatment on the wells (coating layer formation), polyvinyl alcohol having azide groups at a side chain as a water-soluble resin (manufactured by Toyo Gosei Co., Ltd., AWP (Azide-unit pendant Water-soluble Photopolymer, r1=1 to 1000, r2=4 to 4995, r3=0 to 4000, n=1, 2, or 3, and R is a group represented by Formula (III)): Compound represented by Formula (IIa) (average degree of polymerization of water-soluble resin 1600, photosensitive group introduction rate 0.65 mol %)) was dissolved in a 25% by volume aqueous ethanol solution in a light-shielded polypropylene container colored with a brown pigment and a 0.5% by weight water-soluble resin solution was prepared.

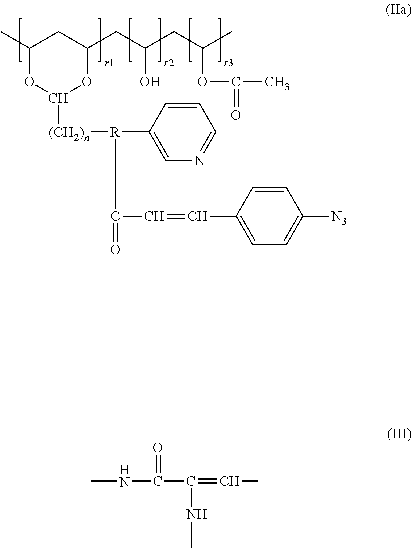

50 μL of the above 0.5% by weight water-soluble resin solution was added per well to a plasma-treated multi-well plate and allowed to stand for 1 minute, then the plate was turned over to discard the excess solution. Next, after primary drying at 40° C. for 60 minutes, the water-soluble resin was cured and cross-linked by irradiation with 250 nm UV light at 1.0 mW/cm²×30 seconds using a UV lamp. Next, the multi-well plates were then washed three times repeatedly with ultrapure water, dried, and then sterilized by irradiation with γ-rays such that the absorbed dose was 22.5 kGy or 45.0 kGy, thus obtaining two types of sterile containers for cell culture.

A cell suspension of HepG2 dispersed in a liquid culture medium (Dulbecco's modified Eagle's medium (DMEM) containing fetal bovine serum (FBS)) was prepared and dispensed into the wells of the two types of sterile containers for cell culture obtained above to obtain the number of cells of 1×10³ cells/well and cell culture was performed at 37° C. for 3 days.

As a result, it was confirmed under a microscope that one cell aggregate (a spheroid) having a diameter with a size of approximately 700 μm was formed in all wells of both sterile containers for cell culture.

### Example 2

Petri dish-shaped containers (volume: 6.6 mL) having a diameter of 90 mm and a height of 1.16 mm were subjected to a plasma treatment and a water-soluble resin treatment using the same method as in Example 1 and then subjected to the same UV irradiation and radiation sterilization (γ-ray irradiation). The absorbed doses of γ-rays were set to 15.0 kGy, 25.0 kGy, or 40.0 kGy to obtain three types of sterile containers for cell culture.

Then, pure water was accommodated in the cell accommodation parts of these three types of sterile containers for cell culture to be 0.12 mL/cm² as the amount with respect to the surface area of the region where the coating layer was formed and heating was carried out at 37° C. for 72 hours, the obtained test solution was placed in a cell with a layer length of 1 cm, and the absorbance measured by irradiation with UV light at a wavelength of 220 nm and the absorbance measured by irradiation with UV light at a wavelength of 241 nm were measured using a UV spectrophotometer. The results are shown in Table 1 below.

The results showed that both absorbances were 0.10 or less, indicating that there was little eluted material in the test solution. In other words, sterile containers for cell culture with γ-ray absorbed doses of 25.0 kGy and 40.0 kGy have almost the same level of eluted material as a sterile container for cell culture with a γ-ray absorbed dose of 15.0 kGy and thus it was clear that, even after being subjected to strong radiation sterilization, the decomposition of the coating layer was small and the coating layer was stably maintained.

TABLE 1
Elution conditions: water, 37° C., 72 h, 6.6 mL/57 cm²
Materials: 90 mm petri dish-shaped sterile container for cell culture
(manufactured by Toyo Gosei Co., Ltd., AWP coating layer present)

| | Absorbance of | Absorbance of | Absorbance of |
|---|---|---|---|
| Measurement | sterile product | sterile product | sterile product |
| wavelength | at 15 kGy | at 25 kGy | at 40 kGy |
| 220 nm | 0.06 | 0.07 | 0.09 |
| 241 nm | 0.03 | 0.04 | 0.04 |

### REFERENCE SIGNS LIST

100 Sterile container for cell culture (multi-well plate)
11 Cell accommodation part (well)
11a Inner surface of cell accommodation part (well)
21 Coating layer
31 Cell aggregate
41 Cross-linked structure (in water-soluble resin)
43 Cross-linked structure (between water-soluble resin and inner surface)

What is claim is:

1. A sterile container for cell culture having a low cell adsorption property, comprising:
a cell accommodation part which is made of a resin material and accommodates cells and a liquid culture medium to perform cell culture,
wherein a coating layer made of a water-soluble resin having a hydrophilic functional group at a side chain is formed on an inner surface of the cell accommodation part, cross-linking is formed in the water-soluble resin itself and between the water-soluble resin and the inner surface of the cell accommodation part, and
the container is subjected to a radiation sterilization treatment with an absorbed radiation dose of 20 kGy or more and 45 kGy or less.

2. The sterile container for cell culture according to claim 1,
wherein, when pure water is accommodated in the cell accommodation part to be 0.12 mL/cm², heating is carried out at 37° C. for 72 hours, and an obtained test solution is placed in a cell with a layer length of 1 cm, an absorbance measured by irradiation with UV light at a wavelength of 220 nm and an absorbance measured by irradiation with UV light at a wavelength of 241 nm are both 0.10 or less.

3. The sterile container for cell culture according to claim 1,
wherein, when human hepatocellular carcinoma-derived cells (HepG2) are accommodated in each of 96 cell accommodation parts along with Dulbecco's modified Eagle's medium (DMEM) containing fetal bovine serum (FBS), the number of human hepatocellular carcinoma-derived cells (HepG2) in each of the cell accommodation parts is 1×10³ cells, and cell culture is performed at 37° C. for 3 days, the number of cell accommodation parts in which one or more cell aggregates having a diameter of 200 μm or more of the human hepatocellular carcinoma-derived cells (HepG2) are formed is 88 or more.

4. The sterile container for cell culture according to claim 1,
wherein the water-soluble resin forming the coating layer has a side chain including an aromatic azide group and a side chain including a carbonyl group,
a carbonyl group is formed in the inner surface of the cell accommodation part, and
cross-linking is formed between the aromatic azide group of the water-soluble resin and the carbonyl group of the water-soluble resin and between the aromatic azide group of the water-soluble resin and the carbonyl group in the inner surface of the cell accommodation part.

5. The sterile container for cell culture according to claim 1,
wherein the water-soluble resin forming the coating layer is a water-soluble resin represented by Formula (Ia) or Formula (Ib), 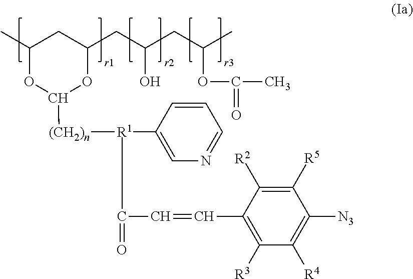
(in Formula (Ia), R1 is a hydrocarbon group having a carbonyl and an amine, R2, R3, R4, and R5 are all hydrogen or alkyl groups, r1 indicates 1 to 1000, r2 indicates 40 to 4995, r3 indicates 0 to 4000, and n indicates 1, 2, or 3), 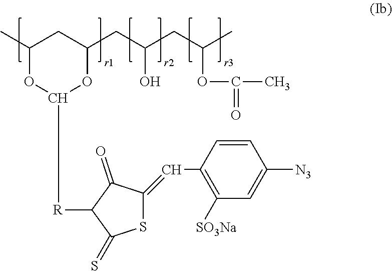
(in Formula (Ib), R indicates a hydrocarbon group having a carbonyl and an amine, r1 indicates 1 to 1000, r2 indicates 40 to 4995, and r3 indicates 0 to 4000).

6. The sterile container for cell culture according to claim 1,
wherein the cell accommodation part is a well and the sterile container for cell culture is a multi-well plate provided with a plurality of the wells.

7. A sealed and packaged sterile container for cell culture,
wherein the sterile container for cell culture according to claim 1 is sealed and packaged using two or more layers using a film material having a gas permeability.

8. A sterile container for cell culture having a low cell adsorption property, comprising:
a cell accommodation part which is made of a resin material and accommodates cells and a liquid culture medium to perform cell culture,
wherein a coating layer made of a water-soluble resin having a hydrophilic functional group at a side chain is formed on an inner surface of the cell accommodation part,
cross-linking is formed in the water-soluble resin itself and between the water-soluble resin and the inner surface of the cell accommodation part,
when pure water is accommodated in the cell accommodation part to be 0.12 mL/cm², heating is carried out at 37° C. for 72 hours, and an obtained test solution is placed in a cell with a layer length of 1 cm, an absorbance measured by irradiation with UV light at a wavelength of 220 nm and an absorbance measured by irradiation with UV light at a wavelength of 241 nm are both 0.10 or less, and
when human hepatocellular carcinoma-derived cells (HepG2) are further accommodated in each of 96 cell accommodation parts along with Dulbecco's modified Eagle's medium (DMEM) containing fetal bovine serum (FBS), the number of human hepatocellular carcinoma-derived cells (HepG2) is 1×10³ cells in each of the cell accommodation parts, and cell culture is performed at 37° C. for 3 days, the number of cell accommodation parts in which one or more cell aggregates having a diameter of 200 μm or more of the human hepatocellular carcinoma-derived cells (HepG2) are formed is 88 or more.
